(19) United States
(12) Patent Application Publication
(10) Pub. No.: US 2025/0195061 A1
(43) Pub. Date: Jun. 19, 2025

(54) METHODS OF TREATING OR REPAIRING TISSUE TRANSLUMINALLY

(71) Applicant: BOSTON SCIENTIFIC SCIMED, INC., MAPLE GROVE, MN (US)

(72) Inventors: Bu'Hussain Hayee, Maple Grove, MN (US); Jamie Kelly, Maple Grove, MN (US)

(73) Assignee: BOSTON SCIENTIFIC SCIMED, INC., MAPLE GROVE, MN (US)

(21) Appl. No.: 18/980,241

(22) Filed: Dec. 13, 2024

Related U.S. Application Data
(60) Provisional application No. 63/610,793, filed on Dec. 15, 2023.

(51) Int. Cl. A61B 17/04; A61B 17/00

(52) U.S. Cl. CPC A61B 17/0469; A61B 2017/00827

(57) ABSTRACT

Various methods of performing a minimally-invasive procedure within a patient using a device and system capable of retroflexing more than 90°, such as at least up 210°. In some aspects, the methods include restoring and/or reshaping an anatomical structure not previously readily accessible via minimally-invasive procedures. The methods also include treating gastroesophageal reflux disease, such as by repairing or creating a gastroesophageal flap valve, and/or by restoring or forming an acute angle of HIS between the esophagus and stomach.

### CROSS-REFERENCE TO RELATED APPLICATIONS

This application claims the benefit of priority under 35 U.S.C. § 119 to U.S. Provisional Application No. 63/610,793, filed Dec. 15, 2023, the entire disclosure of which is hereby incorporated by reference herein for all purposes.

### FIELD

The present disclosure relates to methods for performing a procedure within a patient's body. More particularly, the present disclosure relates to methods for treating or repairing tissue transluminally within a patient's body. Even more particularly, the present disclosure relates to methods performed upon retroflexing a medical device within a patient, such as retroflexing a distal end of the device greater than 180°.

### BACKGROUND

Various procedures may be performed within a body transluminally, such as through a natural orifice or body passage. Optionally, transluminal procedures are performed by advancing and/or retracting a medical device through a tubular member inserted into the patient's body, such as through a natural orifice, a small cut (e.g., laparoscopically), a body passage (e.g., a tortuous body passage), etc. The tubular member typically is a flexible tubular elongate member capable of being navigated within a patient's body, including through curved and/or tortuous body passages. Flexible tubular elongate members which may be used include medical scopes, such as endoscopes. In order to navigate within the patient's body, and to facilitate access to a target site at which a procedure is to be performed, flexible tubular elongate members may include various mechanisms, such as internal pulleys and cables, to steer and/or navigate the member through a patient's body (including tortuous body passages). For instance, some medical scopes may be articulated in one or more planes, such as for up-down movement and/or left-right movement. There are times when the ability of a flexible tubular elongate member to flex is limited either by the limits of maximum flexion of the member, and/or by diminished performance of the member over time. Moreover, the bulk added by various additional auxiliary devices which may be extended through or along the flexible tubular elongate member scope may also affect articulation of the flexible tubular elongate member/overall system. The resulting difficulty in maneuvering the flexible tubular elongate member used to access and perform a procedure within a patient has resulted in various constraints on the procedures which may be performed. For instance, a minimally invasive approach to repairing anatomical structures to treat gastroesophageal reflux disease typically requires flexing and retroflexing of the surgical devices and systems beyond what is typically achievable by existing devices and systems. It is with respect to these and other considerations that the present improvements may be useful.

### SUMMARY OF THE DISCLOSURE

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a method of treating gastroesophageal reflux disease in a patient includes inserting a medical device through the esophagus of the patient and into the stomach of the patient; retroflexing a distal end of the medical device to access a region of the stomach adjacent the gastroesophageal junction; and modifying the structure of the region of stomach adjacent the gastroesophageal junction to create a structure which reduces the patient's gastroesophageal reflux disease.

In some aspects, the medical device is a suturing device, and the method further includes suturing a region of the stomach adjacent the gastroesophageal junction. In some aspects, the method further includes applying a line of sutures in an anterior-posterior direction and at least one line of sutures in a lateral-medial direction. In some aspects, applying at least one line of sutures in a lateral-medial direction further includes applying a first line of sutures in a lateral-medial direction along a posterior region of the patient's stomach, and a second line of sutures in a lateral-medial direction along an anterior region of the patient's stomach. In some aspects, applying at least one line of sutures in a lateral-medial direction further includes applying at least one line of sutures from a lateral region of the patient's stomach in a direction towards the medial region of the patient's stomach. In some aspects, the first line of sutures in a lateral-medial direction is applied before the second line of sutures in a lateral-medial direction is applied. In some aspects, applying the line of sutures in an anterior-posterior direction further includes applying the line of sutures in an anterior-posterior direction along a lateral region of the patient's stomach. In some aspects, applying the line of sutures in an anterior-posterior direction further includes applying the line of sutures in an anterior-posterior direction along a lateral region of the patient's stomach.

In accordance with various principles of the present disclosure, a method of suturing a patient's stomach to restore the angle of HIS between the patient's stomach and esophagus includes inserting a medical device through the esophagus of the patient and into the stomach of the patient; retroflexing a distal end of the medical device to access a region of the stomach adjacent the gastroesophageal junction; and modifying the structure of the region of stomach adjacent the gastroesophageal junction to create a structure which reduces the patient's gastroesophageal reflux disease.

In some aspects, the medical device is a suturing device, and the method further includes suturing a region of the stomach adjacent the gastroesophageal junction. In some aspects, the method further includes applying a line of sutures in an anterior-posterior direction and at least one line of sutures in a lateral-medial direction. In some aspects, applying at least one line of sutures in a lateral-medial direction further includes applying a first line of sutures in a lateral-medial direction along a posterior region of the patient's stomach, and a second line of sutures in a lateral-medial direction along an anterior region of the patient's stomach. In some aspects, applying at least one line of sutures in a lateral-medial direction further includes applying at least one line of sutures from a lateral region of the patient's stomach in a direction towards the medial region of the patient's stomach. In some aspects, the first line of sutures in a lateral-medial direction is applied before the second line of sutures in a lateral-medial direction is applied. In some aspects, applying the line of sutures in an anterior-posterior direction further includes applying the line of sutures in an anterior-posterior direction along a lateral region of the patient's stomach. In some aspects, applying the line of sutures in an anterior-posterior direction further includes applying the line of sutures in an anterior-posterior direction along a lateral region of the patient's stomach.

In accordance with various principles of the present disclosure, a method for accessing the gastroesophageal junction of a patient's stomach includes inserting a medical device through the esophagus of the patient and into the stomach of the patient; and retroflexing a distal end of the medical device to access the gastroesophageal junction.

In some aspects, the method further includes axially rotating the medical device to access different sides of the gastroesophageal junction.

In some aspects, the medical device includes a medical scope, and the method further includes visually accessing the gastroesophageal junction of the patient by viewing the gastroesophageal junction with the optical system of the medical scope. In some aspects, the method further includes advancing a medical device in addition to the medical scope along the medical scope to access the gastroesophageal junction of the patient.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, or shrunk so that structural and/or anatomical context may be included. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a side elevational view of a suturing device which may be used with a method in accordance with various principles of the present disclosure,

FIG. 2 is a side elevational view of a suturing device as in FIG. 1 in a retroflexed configuration.

FIG. 3 is a schematic illustration of a stomach and adjacent portion of an esophagus with a suturing device such as illustrated in FIG. 2 inserted therein.

FIG. 3A is cross-sectional view along line IIIA-IIIA in FIG. 3.

FIG. 4 is a schematic elevational view of a stomach before suturing in accordance with various principles of the present disclosure has been performed.

FIG. 4A is a cross-sectional view along line IVA-IVA in FIG. 4.

FIG. 5 is a schematic elevational view of a stomach after suturing in accordance with various principles of the present disclosure has been performed.

FIG. 5A is a cross-sectional view along line VA-VA in FIG. 5.

FIG. 6 illustrates planimetry readings before and after suturing in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. As understood herein, “corresponding” is intended to convey a relationship between components, parts, elements, etc., configured to interact with or to have another intended relationship with one another. Finally, reference to “at” a location or site is intended to include at and/or about the vicinity of (e.g., along, adjacent, proximate, etc.) such location or site.

Among the various minimally-invasive (e.g., transluminal) procedures which are known and being developed or further developed, anti-reflux endoscopic therapies (ARETs) are of increasing interest. Gastroesophageal reflux disease (GERD) is a highly prevalent global phenomenon, affecting nearly 10,000 per 100,000 of the population, or up to 14% when complications are also taken into account. Diet and lifestyle changes, medication, and/or surgery are the most prevalent treatment options. Minimally-invasive anti-reflux endoscopic therapies are typically aimed at restoring or repairing the “normal” anti-reflux anatomy. For instance, ARETs may be performed to repair the gastroesophageal flap valve associated with the lower esophageal sphincter and/or to repair the angle of HIS (also known as the esophagogastric angle, along the upper exterior of the stomach). Currently available approaches utilize devices such as bespoke stapling and/or suturing devices aimed at repairing, reshaping, restoring, etc., the anatomy of the esophagus and/or stomach to a configuration (a “natural” configuration) in which gastric materials (particularly stomach acid) are prevented from moving from the stomach to the esophagus. For instance, a weakened lower esophageal sphincter (i.e., LES, also known as the cardiac sphincter, gastroesophageal sphincter, or gastroesophageal junction), a hiatal hernia, improper closure of the gastroesophageal flap valve (also known as the anti-reflux “flap valve”), and other structural anatomical issues may allow reflux to occur. Correction of such structural anatomical issues to prevent gastric materials from entering the esophagus is one approach to correcting GERD, particularly severe GERD.

There has been a growing interest in minimally-invasive procedures, e.g., transluminal, transcatheter, endoscopic, etc., procedures which do not require open surgery (cutting open the patient), but, instead, access a target site within the patient via a natural orifice (or, in some instances, a small incision not considered to constitute an open-surgery cut). Various devices and systems have been used to correct anatomical structural issues associated with GERD with a minimally-invasive procedure. For instance, various well-established, procedure-agnostic platforms, designed for endoscopic placement of anchors, sutures, anchor-sutures, surgical staples, etc., may be used to surgically correct various gastric or gastroesophageal anatomies via oral access. However, various mechanical limitations on such platforms have thus far inhibited full transluminal access to various gastroesophageal structures to be operated upon to address GERD. For instance, typical devices and systems are not capable of the degree of flexion and/or retroflexion necessary to engage various target sites such as the gastroesophageal junction.

Methods in accordance with various principles of the present disclosure utilize a retroflexion device and system configured for mounting with respect to a flexible elongate member with associated tools for repairing anatomical structures to articulate and retroflex the flexible elongate member and/or the associated tools. It will be appreciated that terms such as mount, engage, receive, couple, connect, attach, associate with, operatively associate with, etc., including other grammatical forms thereof, may be used interchangeably herein without intent to limit unless otherwise indicated. It will further be appreciated that, as used herein, retroflexion (including various grammatical forms thereof) is intended to refer to increased flexibility and bending of a device, such as to bend such device backwards, e.g., beyond 90° from its initial direction. In other words, a distal end of a generally longitudinally extending device extends distally, but may be retroflexed to extend in a proximal direction. The increased flexibility and degree of articulation which may be achieved by the flexible elongate member with the aid of a retroflexion device and system utilized in accordance with various principles of the present disclosure allows increased access to anatomical sites within a body, particularly during transluminal and/or endoscopic procedures in which the patient's body is accessed in a minimally invasive manner (i.e., without cutting open the patient's body surgically). Moreover, use of a retroflexion device and system allowing a degree of retroflexion not previously achievable allows the flexible elongate member greater access to the patient's anatomy than previously afforded or achievable by prior devices and systems to allow development and performance of various methods in accordance with various principles of the present disclosure.

In accordance with various principles of the present disclosure, methods involving more versatile and robust suturing procedures are performed to achieve minimally-invasive surgical results not previously achievable without more invasive surgery. More particularly, a new surgical platform, which has recently become available, provides a device and system which may be flexed or retroflexed greater than 90° and even greater than 180°, such as up to about 210° and in some instances even further. Such extended range of movement of a minimally-invasive surgical device and/or system allows procedures disclosed herein to be performed, and which could not previously be contemplated. More particularly, in accordance with various principles of the present disclosure, a device and system are retroflexed to perform suture plication in retroflexion in a novel anti-reflex endoscopic therapy (ARET) which may be referenced herein as anti-reflux endoscopic suturing (ARES).

In some aspects, a method in accordance with various principles of the present disclosure utilizes a retroflexion device and system associated with a flexible elongate member in a manner allowing the retroflexion device and system to articulate and retroflex a distal end of the flexible elongate member. The retroflexion device may be mounted with respect to the flexible elongate member as part of a system which includes additional components. In some aspects, the flexible elongate member is a medical scope. In some aspects, various tools, instruments, devices, etc. (such terms being used interchangeably herein without intent to limit) are operatively associated with the flexible elongate member. For instance, in some aspects, the flexible elongate member is a medical scope, and one or more devices are advanced/retracted/operated through a working channel of the medical scope. In some aspects, various devices are advanced/retracted, operate along the exterior of the medical scope. In some aspects, the devices extend along/alongside the exterior of the medical scope. In some aspects, the devices are mounted on (e.g., on the exterior of) a medical scope, such as at or along a distal end of the medical scope (e.g., on an end cap mounted on the distal end of the medical scope). In some aspects, the devices are configured for performing a procedure such as a suturing/suture plication procedure in accordance with various principles of the present disclosure.

Various anti-reflux endoscopic therapies, such as anti-reflux endoscopic suturing therapies, in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, steps, and/or characteristics, or that an embodiment includes all features, structures, concepts, steps, and/or characteristics. Some embodiments may include one or more such features, structures, concepts, steps, and/or characteristics, in various combinations thereof. It should be understood that one or more of the features, structures, concepts, steps, and/or characteristics described with reference to one embodiment can be combined with one or more of the features, structures, concepts, steps, and/or characteristics of any of the other embodiments provided herein. That is, any of the features, structures, concepts, steps, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, steps, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, steps, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

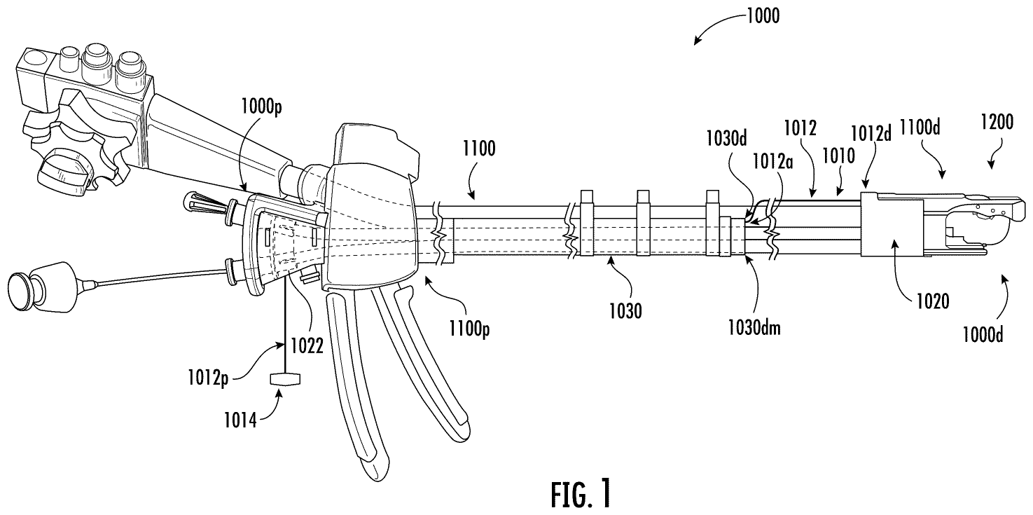

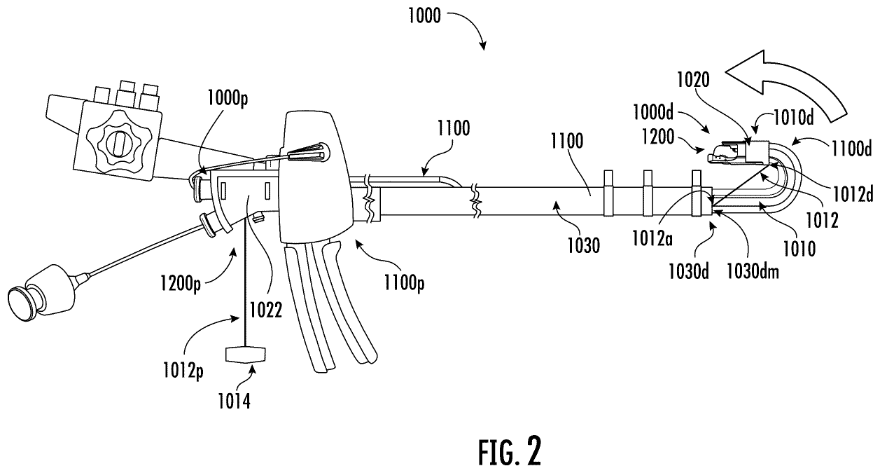

Turning now to the drawings, an example of an embodiment of a retroflexion system 1000 used to perform methods (which previously had not been feasible) in accordance with various principles of the present disclosure is illustrated in FIG. 1 and FIG. 2. The illustrated retroflexion system 1000 includes a retroflexion device 1010 operatively associated with a flexible elongate member 1100. The retroflexion device 1010 includes an actuation component 1012 which may be referenced herein, for the sake of convenience and without intent to limit, as a pull string 1012. The pull string 1012 of the system 1000 illustrated in FIG. 1 and FIG. 2 is an elongate element having sufficient strength to be actuated from a proximal end 1012p thereof to cause a distal end 1012d thereof to actuate the flexible elongate member 1100 with which the retroflexion device 1010 is operatively associated. The flexible elongate member 1100 may be a component of a medical device, or may reference a medical device in general, such as a medical scope. Reference is generally made herein to a medical scope, however methods of the present disclosure need not be limited in this regard. Articulation and/or retroflexion of the flexible elongate member 1100 by the retroflexion system 1000 of the present disclosure may advantageously direct a distal working end, or the opening of a working channel (e.g., of a medical scope) to a target site within a patient's body. The working end of a medical device (either the flexible elongate member 1100, if in the form of a medical device, or a medical device extending through a working channel of a flexible elongate member in the form of a medical scope) may thereby be directed more accurately to the target site than previously achievable by prior art systems and devices.

To effect retroflexion of the system 1000 illustrated in FIG. 1 and FIG. 2, a pull handle 1014 may be provided along the proximal end 1012p of the pull string 1012. In some aspects, the proximal end 1012p of the pull string 1012 may be operatively associated with an adaptor/proximal mount 1022 provided along a proximal end 1000p of the retroflexion system 1000, such as along a proximal end 1030p of a flexible tubular elongate member 1030 along which the pull string 1012 of the retroflexion device 1010 extends distally. The distal end 1012d of the pull string 1012 is operatively associated with the flexible elongate member 1100 adjacent a section of the flexible elongate member 1100 which is to be articulated by the retroflexion system 1000. In the example of an embodiment illustrated in FIG. 1, the retroflexion system 1000 includes a distal mount 1020 having a section thereof configured to be engaged with the flexible elongate member 1100. In some aspects, the distal mount 1020 may be a collar which may encircle and/or be affixed to (tightened, adhered, clamped, etc., with respect to) the flexible elongate member 1100. In some embodiments, the distal mount 1020 is an end cap configured for mounting with respect to a distal end of a medical scope such as an endoscope. In some aspects, the distal mount 1020 is in the form of an end cap includes indicia for aligning with respect to a medical scope so as not to interfere with various functionalities or features of the scope, such as an optical system (e.g., visualization systems such as a camera, optical fiber, etc.), or working channels thereof.

In the example of an embodiment illustrated in FIG. 1, the distal end 1012d of the pull string 1012 is operatively associated with the flexible elongate member 1100 via the distal mount 1020 so that when the pull string 1012 is pulled proximally (e.g., by pulling on a proximal end 1012p thereof), the distal end 1012d pulls the distal mount 1020 proximally to flex the flexible elongate member 1100 proximally (e.g., in the direction of the illustrated flexion arrow F), such as illustrated in FIG. 2. In order to cause flexing of the flexible elongate member 1100 (e.g., the distal end 1100d thereof), an actuation region 1012a of the pull string 1012 proximal to the distal end 1012d thereof is held against lateral movement (e.g., anchored) with respect to the flexible elongate member 1100 (while still being longitudinally movable along the flexible elongate member 1100). The actuation region 1012a may provide a pivot point for the pull string 1012 to flex the flexible elongate member 1100. In some aspects, the pivot mount which holds the actuation region 1012a of the pull string 1012 with respect to the flexible elongate member 1100 may be the distal end 1030d of a flexible tubular elongate member 1030 (e.g., a sheath) extending along the flexible elongate member 1100 and defining a lumen therethrough through which the pull string 1012 translates/movably extends. In some aspects, the distalmost end 1030dm of the flexible tubular elongate member 1030 may provide an anchor or pivot point for the actuation region 1012a of the pull string 1012, such as by serving as a pivot mount, and the distance between the distal end 1012d of the pull string 1012 and the actuation region 1012a may influence the bending radius of the flexed region of the flexible elongate member 1100.

When a retroflexion system 1000 such as described herein is used in conjunction with a flexible elongate member 1100 in the form of a medical scope, the retroflexion system 1000 adds further degrees of movement to a pre-existing medical scope, and thereby may enhance articulation and/or retroflexion thereof and/or of any instruments used therewith (e.g., advanced through a working channel thereof, or otherwise operatively associated with the medical scope). For instance, in some embodiments, a retroflexion system 1000 such as described herein may allow the flexible elongate member 1100 operatively associated therewith to flex greater than about 90°, such as greater than about 145°, such as greater than about 180°, and up to at least or even greater than about 210° (including various increments therebetween, such as increments of 1°). Optionally, additional devices may be used in conjunction with and/or independently of the flexible elongate member 1100, to perform novel medical procedures in accordance with various principles of the present disclosure. Additional devices may be extended along the exterior of the flexible elongate member 1100, and either also along the retroflexion device 1010, or through a lumen through the flexible tubular elongate member 1030 through which the pull string 1012 of the retroflexion device 1010 may extend. Additionally or alternatively, additional devices may extend through a lumen defined through the flexible elongate member 1100 with which the retroflexion system 1000 is associated, such as through a working channel of a flexible elongate member 1100 in the form of a medical scope.

As may be appreciated, as the distal portions 1000d, 1100d of the retroflexion system 1000 and flexible elongate member 1100 are retroflexed, such distal portions 1000d, 1100d approach the portions of the retroflexion system 1000 and flexible elongate member 1100 proximal to the bent portions thereof. However, various properties of the retroflexion system 1000 and flexible elongate member 1100 (or at least the distal portions 1000d, 1100d thereof), such as stiffness, resiliency, bendability, etc., typically will create a bending radius which spaces the distal portions 1000d, 1100d of the retroflexion system 1000 and flexible elongate member 1100 from the portions proximal thereto as the retroflexion system 1000 and flexible elongate member 1100 are retroflexed. Such spacing typically allows the distal portions 1000d, 1100d to be retroflexed greater than 180° since the spacing typically prevents the portions of the retroflexion system 1000 and flexible elongate member 1100 which are proximal to the distal portions 1000d, 1100d from physically interfering with retroflexion. In some aspects, the distal portions 1000d, 1100d of the retroflexion system 1000 and flexible elongate member 1100 are capable of being retroflexed greater than 180°, and even as much as 210° before contacting the portions of the retroflexion system 1000 and flexible elongate member 1100 proximal thereto. Such retroflexion is greater than previously achievable by existing devices and allows the flexible elongate member 1100 to access a greater range of target sites. It will be appreciated that the closer the distal portions 1000d, 1100d of the retroflexion system 1000 and flexible elongate member 1100 are bent toward the proximal portions of the retroflexion system 1000 and flexible elongate member 1100, the more the distalmost ends of the retroflexion system 1000 and flexible elongate member 1100 face the proximal portions, and the more the proximal portions may interfere with use of the distal portions 1000d, 1100d.

In some aspects, such retroflexion allows access to target sites which are not along the anatomical passageway through which the flexible elongate member 1100 is inserted (e.g., not distal or otherwise longitudinally along the insertion path of the flexible elongate member 1100). More particularly, if the flexible elongate member 1100 is inserted through an anatomical passageway or structure in a first direction, and the target site is not aligned with that first direction (e.g., is positioned 45° or 90° or more from a path or direction of insertion or advancement of the flexible elongate member 1100), the distal end 1100d of the flexible elongate member 1100 must be flexed to access the target site, but various constraints on prior systems and devices have interfered with achieving the necessary degree of flexion. A system 1000 as described herein allows for methods described herein by allowing access to the fundus of a patient's stomach with a medical scope inserted through a patient's mouth and esophagus which had not been previously achievable. Various procedures are thus performed in accordance with various principles of the present disclosure, such as GERD repair in the region of the fundus, which had been difficult if not impossible to be performed endoscopically, transluminally, etc.

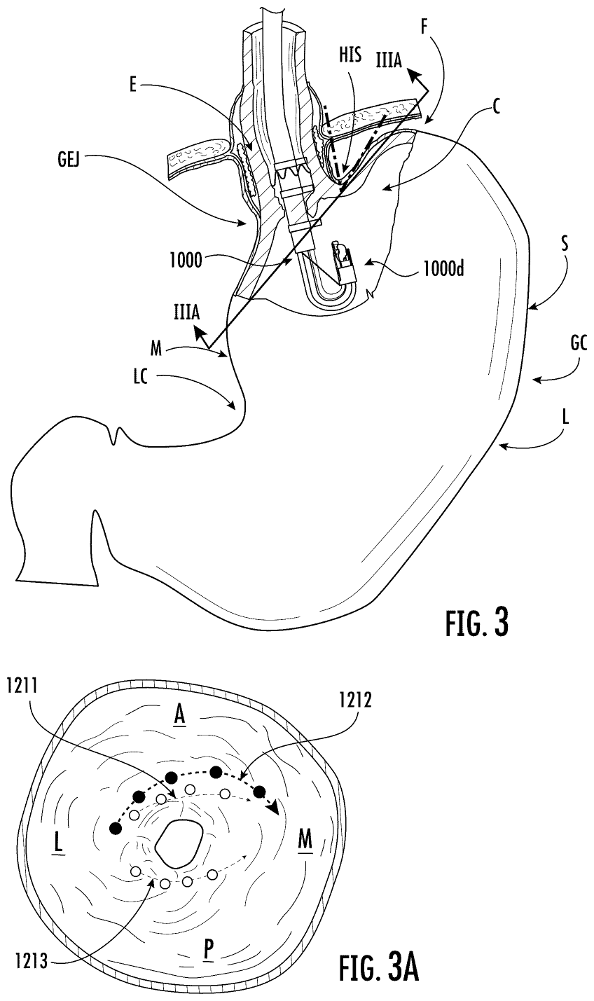

As illustrated in the schematic representation of an esophagus E and stomach S in FIG. 3, a retroflexion system 1000 used with methods of the present disclosure allows sufficient retroflexion such that when advanced through a patient's esophagus E into the patient's stomach S, the distal end 1000d of the retroflexed component of the system 1000 may be sufficiently retroflexed into a direction generally aligned with the cardia C and the fundus F of the stomach. Procedures in such regions of the stomach, such as GERD repair, which had previously been difficult to perform may be performed with the use of a retroflexed component of a retroflexion system 1000 such as described above.

As noted above, a retroflexion system 1000 used with methods of the present disclosure may include one or more devices for performing a medical/surgical procedure with respect to an anatomical structure of a patient. In the example of an embodiment of a system 1000 illustrated in FIG. 1 and FIG. 2, one such device is a suturing device 1200 which is mounted with respect to the distal end 1100d of the flexible elongate member 1100. In the illustrated example of an embodiment, the suturing device 1200 is mounted on or distal to the distal mount 1020 which, in turn, is mounted on/over a distal end 1100d of the flexible elongate member 1100. As such, the suturing device 1200 illustrated in FIG. 1 and FIG. 2 is not a through-the scope device, but, instead, is mounted over, external to the working channel(s) of the scope used with the system 1000. However, methods of the present disclosure need not be limited in this regard. Further details of examples of embodiments of a device in the form of a suturing device mounted with respect to the flexible elongate member 1100 of a system 1000 used in accordance with various principles of the present disclosure are provided in U.S. Pat. No. 9,198,562 to Mitelberg et al., issued on Dec. 1, 2015; U.S. Pat. No. 9,867,610 to Mitelberg et al., issued on Jan. 16, 2018; U.S. Pat. No. 11,051,800 to Mitelberg et al., issued Jul. 6, 2021, or Patent Application Publication Number 2024/0366213, filed on May 3, 2024, and published on Nov. 7, 2024, the entire contents of which are incorporated herein by reference for all purposes. Furthermore, it will be appreciated that additional movable or fixed devices may be used with a system 1000 or method of the present disclosure, in addition to or instead of the suturing device 1200.

A retroflexion system 1000 utilized by methods of the present disclosure allows a suturing device 1200 such as illustrated in FIG. 1 and FIG. 2 to be positioned to reach various anatomical structures not readily reachable with previous systems. In accordance with various principles of the present disclosure, a method for restoring and/or repairing an anatomical structure and/or treating a condition by surgically affecting an anatomical structure is performed using a retroflexion system 1000 such as illustrated in FIG. 1 and FIG. 2 (although the methods need not be limited in this regard). For instance, as illustrated in FIG. 3, a retroflexion system 1000 such as described herein may be inserted (e.g., orally) through a patient's esophagus E and into the patient's stomach S, and then may be retroflexed, as illustrated, to allow a distal working end 1000d thereof to access the gastroesophageal junction GEJ of the patient. One or more devices of the retroflexion system 1000 may then be used to repair the gastroesophageal junction GEJ of a patient, such as to treat GERD, in manners not previously achievable. For instance, a suturing device 1200 of a retroflexion system 1000 of the present disclosure may be used to apply two or more suture lines along a selected area of the anatomical structure to reshape, repair, restore, create, etc., a properly functioning anatomical structure. It will be appreciated that terms such as reshape, repair, restore, create, etc., including other grammatical forms thereof, may be used interchangeably herein without intent to limit.

For instance, a suturing device 1200 of a retroflexion system 1000 formed in accordance with various principles of the present disclosure may be used to suture a region of the stomach S, such as adjacent the gastroesophageal junction GEJ and/or the fundus F, such as to treat GERD. One or more sutures, such as one or more lines of sutures (i.e., multiple sutures in series, either linearly or nonlinearly arranged) may be applied to one or more regions typically not previously accessible by prior systems to achieve surgical results not previously achievable. In some aspects, the sutures applied by the retroflexion system 1000 pull the full thickness of the walls of the stomach S. In other words, in some aspects, the needle 1202 of the suturing device 1200 (illustrated in FIG. 1 and FIG. 2) penetrates, from within the stomach, through the gastric serosa (outermost layer of the stomach wall).

An example of a suture pattern which may be formed in the stomach S in accordance with various principles of the present disclosure to repair the gastroesophageal junction GEJ and/or to otherwise treat GERD, is illustrated in FIG. 3A, showing a view along line IIIA-IIIA in FIG. 3, such as adjacent the gastroesophageal junction GEJ and/or the fundus F. The illustrated suture pattern may be used to restore the angle of HIS (indicated schematically in broken lines in FIG. 3) or otherwise to restore the anatomy of the gastroesophageal junction GEJ and/or the fundus F (e.g., to restore the gastroesophageal flap) to result in proper functioning of the gastroesophageal system. More particularly, in the example of an embodiment of a suturing pattern illustrated in FIG. 3A, two or more suture lines 1211, 1212, 1213, each being formed by one or more “bites” of the suture into tissue (e.g., tissue acquisition points at which suture material is passed through the tissue) represented in the drawings as “dots”, may be formed in accordance with various principles of the present disclosure with a suturing device 1200 included with a retroflexion system 1000 such as described above. In some aspects, the bites are full thickness bites which penetrate through, such as fully through, the stomach wall (i.e., from the within the stomach S, through the mucosa, the submucosa, the muscularis, subserosa, and serosa layers of the stomach S). As may be appreciated, suturing of the illustrated region of the stomach S could not be previously achieved endoscopically, at least without a device permitting retroflexion as permitted by a suturing device included with a retroflexion system 1000 such as described above.

In some aspects, the first suture line 1211 to be formed in accordance with a method of the present disclosure is a suture line extending between the lateral region L and the medial region M of the stomach S. It will be appreciated that reference may instead be made to a suture line extending between the greater curvature GC of the stomach S (along the lateral side L of the stomach S) and the lesser curvature LC of the stomach S (along the medial side M of the stomach S). It will further be appreciated that the precise location (e.g., along the lateral region L and/or the medial region M of the stomach S) at which the first and/or last bite of a suture line is placed in accordance with various principles of the present disclosure may be determined by various factors such as the patient's anatomical structure, the condition of the tissue, etc. In some aspects, suturing of the first suture line 1211 is performed from a lateral to medial direction. However, suturing from a lateral to medial direction is also within the scope of the present disclosure. In some aspects, the lateral end of the suture line 1211 is positioned/initiated at the angle of HIS and extended medially and in a generally inferior direction (caudally) along the anterior region A of the stomach S, such as generally along a direction of the sling fibers of the stomach S/cardia C (or a direction in which the sling fibers should extend if not present or not properly positioned in the patient). Such suture line 1211 (and optionally further suture lines as will be described) may draw a portion of the stomach S adjacent the gastroesophageal junction GEJ downward to restore or at least contribute to restoring and/or create (if congenitally not present) the angle of HIS. In some aspects, the first suture line 1211 (and optionally one or more additional suture lines, such as suture lines 1212, 1213) may be considered to reinforce and/or restore and/or replace the sling fibers of the stomach S. Alternatively or additionally, the first suture line 1211 (and optionally one or more additional suture lines, such as suture lines 1212, 1213) may be considered to provide what may be considered artificial sling fibers which may restore/create an angle of HIS and/or otherwise repair and/or create structure resulting in treating the patient's GERD.

One or more additional suture lines 1212, 1213 may then be formed, extending between the medial side M of the stomach S and the lateral side L of the stomach S. In some aspects, the suture line 1212 along the posterior side P of the stomach S (and between the lesser curvature LC and the greater curvature GC) is the next suture line formed after the first suture line 1211 is formed, and then another suture line 1213 along the anterior side A of the stomach S (also extending between the lesser curvature LC and the greater curvature GC of the stomach S) is formed. However, other orders of suturing are within the scope of the present disclosure.

Typically, the suture lines 1211, 1212, 1213 which are formed/placed in accordance with various principles of the present disclosure extend between the greater curvature GC and the greater curvature GC lesser curvature LC, and between the fundus F and a position along the cardia C inferior to the fundus F. Such placement/arrangement/orientation has been found to repair the stomach S, and/or to restore (or at least contribute to restoring) the angle of HIS, and/or to reinforce the sling fibers of the stomach S, thereby treating the patient (e.g., reducing and hopefully eliminating GERD or at least symptoms thereof)

As may be appreciated in view of the above, the suture lines 1211, 1212, 1213 typically draw together and/or reinforce portions the tissue/walls of the stomach S to reshape the stomach S into a more functionally correct configuration. For instance, the suture lines 1211, 1212, 1213 may have a vertical position sufficiently adjacent the intended vertex of the angle of HIS to be recreated/restored/adjusted (to change the angle between the esophagus E and the fundus F from one which may be generally obtuse to generally acute) by the method of the present disclosure such that the pulling together and/or reinforcing of tissue walls by the suture line 1211, 1212, 1213 recreates/restores/adjusts the anatomy of the stomach S as intended. In some aspects, it is desirable for at least one or more of the suture lines 1211, 1212, 1213 to be along stomach S tissue (e.g., the wall of the stomach S), in contrast with at the level of the gastroesophageal junction GEJ (i.e., above the stomach S). In some aspects, the formation of more than one suture line in accordance with various principles of the present disclosure results in the desired reconfiguring of the gastroesophageal junction GEJ than would occur with a single suture line (such as a purse-string suture line which may extend continuously along the anterior, medial, and posterior sides of the stomach S). In some aspects, one or more suture lines may be placed between the anterior region A and the posterior region P of the stomach S, the medical professional confirming the anatomical changes created by such suture line do not alter the proper functioning of the gastroesophageal junction GEJ. In some aspects, the anterior-posterior suture line is placed along the greater curvature GC, with the lesser curvature LC, at least to some extent, not being sutured, as reducing suture plication in such region minimizing the possibility of the procedure impeding flow through the esophagus E. In some aspects, a reverse arrangement, with an anterior-posterior suture line along the lesser curvature LC and no suture line along greater curvature GC, may result in the desired structural anatomical changes to the stomach S.

It will be appreciated that the above description of placement, and the above-described order of formation of suture lines 1211, 1212, 1213 is just one example of a manner of applying sutures in accordance with various principles of the present disclosure using a retroflexion system 1000 as described herein. The order in which the suture lines 1211, 1212, 1213 are formed, as well as the direction in which the sutures are formed along a given suture line (e.g., anterior-to-posterior or posterior-to-anterior; medial-to-lateral or lateral-to-medial) may be determined based on case of access, viewability, or other parameters relevant to performing such procedure. For instance, if the flexible elongate member 1100 is a medical scope, different manufacturers arrange the visualization elements (e.g., camera) and/or working channels thereof differently, which may affect accessibility, viewability, etc., of the region of the stomach S at which sutures are to be formed/placed. The medical professional performing the procedure with a retroflexion system 1000 such as described herein may determine the precise placement, direction of suturing, sequence of forming the suture lines, amount of tightening of the suture lines, etc., based on various factors including various characteristics of the anatomy on which the procedure is being performed (e.g., the nature and/or function of the structure to be modified as well as surrounding anatomies, the responsiveness of the tissue, the physical properties of the tissue, etc.) and/or characteristics of the retroflexion system 1000 and suturing device 1200 (e.g., placement and/or orientation of components thereof), as may be appreciated by one of ordinary skill in the art. For instance, not all stomachs have the same anatomical shape, and some stomach shapes are more prone to GERD, so surgical approaches may vary depending on the anatomy presented by the patient. Additionally or alternatively, the configurations of the lines of sutures 1211, 1212, 1213 may be linear, curvilinear, or a combination thereof, such as depending on various considerations such as the shape of the stomach S, the amount of correction to be effected, the condition of the stomach S tissue, etc. Suture lines may be placed in accordance with various principles of the present disclosure with reference to the effect created by each suture bite into the tissue. Thus, the medical professional performs a suture bite, observes the effect of the bite and/or suture line on the tissue/organ, and determines the next step accordingly. Such responsive approach accounts for variations in patient anatomy and tissue characteristics. As may be further appreciated by one of ordinary skill in the art, the opportunity for evaluating such various considerations in performing a method in accordance with various principles of the present disclosure in the first place, to perform such procedures as described herein, was not feasible or available without a retroflexion system 1000 such as described herein, and thus the methods described herein were also not feasible or performable until now.

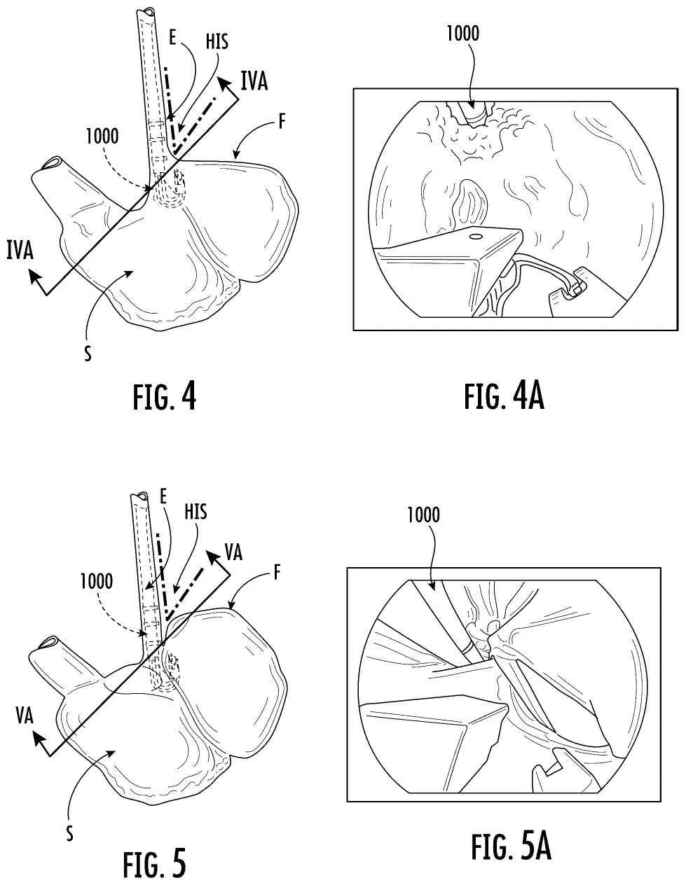

As may be appreciated with reference to FIG. 4 and FIG. 5, a suturing method as described herein reshapes the anatomical structure of the sutured tissue and thus the functioning thereof. More particularly, as illustrated in FIG. 4, the stomach S in the region of the esophagus E/fundus F before suturing in accordance with various principles of the present disclosure has a configuration susceptible and/or predisposed to reflux (the angle of HIS is obtuse instead of acute). In contrast, as may be appreciated with reference to FIG. 5, showing the stomach S of FIG. 4 after suturing in accordance with various principles of the present disclosure, in comparison with FIG. 4, the angle of HIS is restored by a method performed in accordance with various principles of the present disclosure.

As may be further appreciated with reference to FIG. 4A and FIG. 5A, taken along line IVA-IVA in FIG. 4 and line VA-VA in FIG. 5, respectively, in accordance with various aspects of the present disclosure, a method of suturing a stomach may result in reconfiguration of the internal structure of the stomach S, which may further improve functionality of the stomach S (e.g., by reducing GERD). For instance, as may be appreciated with reference to FIG. 5A, suturing such as described herein may result in inward bunching of stomach tissue (which may be appreciated in contrast with tissue prior to suturing, as illustrated in FIG. 4A). Such bunching may result in restoring the angle of HIS and/or in the creation of somewhat of a flap which may reduce reflux of gastric materials from the stomach S into the esophagus E. In some aspects, the bunched tissue and/or flap may support the sphincter at the gastroesophageal junction GEJ. As the stomach muscles contract during the digestive process, pushing the stomach contents towards the esophagus E, the restored angle of HIS, and/or flap or tissue bunching created by the suturing method of the present disclosure, help to deflect stomach contents back into the stomach S. With the structural configuration of the fundus F restored by a method of the present disclosure, some of the stomach contents may be pushed into the generally expandable fundal tissue, which is more stretchy than tissue at the sphincter and is a natural area for food to be directed for recycling during the digestion process within the stomach S. Thus, restoring a functional anatomical shape of the stomach S and/or gastroesophageal junction GEJ is generally considered a well-regarded approach to reducing GERD.

In accordance with various principles of the present disclosure, care must be taken in restoring the anatomical structure of the gastroesophageal junction GEJ and the upper region of the stomach S so as not to create obstructions or other interferences with passing of food through the stomach and into the small intestines. More particularly, the esophagus E and the region between the esophagus E and the stomach S should not be made so narrow as to create potential issues with downward movement of food towards the antrum of the stomach. Any slowing of passage of food downward has the potential of making swallowing more difficult, and potentially causing food to get stuck in the esophagus E. Proper restoration of the structure and function of the esophagus E and the stomach S allows effective swallowing as well as passage of food through the stomach S in a relatively healthy manner without blocking the esophagus E.

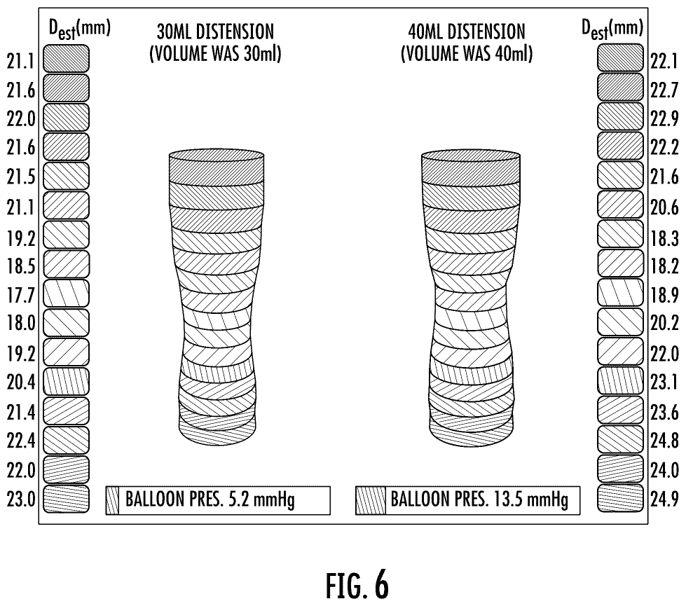

Once sutures are applied in accordance with various principles of the present disclosure, proper functioning may be determined using planimetry to confirm high pressure areas, disruptions, extra narrowing, etc., which could have a deleterious effect on proper gastric functioning, have not been created or caused, and/or have not been alleviated by the suturing. An appropriate measuring device may be inserted into the esophagus E and measurements taken at various levels along the extent of the esophagus E. For instance, an inflatable sensing device may be inserted into the esophagus E and inflated therein (e.g., inflated with fluid such as water or saline). Sensors (e.g., pressure sensors) on the device show pressures at each level along the passage within the esophagus E. Planimetry readings before (at 30 ml balloon distention) and after (at 40 ml balloon distention) suturing in accordance with various principles of the present disclosure are illustrated in FIG. 6. As may be appreciated, suturing in accordance with various principles of the present disclosure restores/maintains positive functionality of the esophagus E so a patient can swallow effectively. Suturing in accordance with various principles of the present disclosure thus restores and/or improves combined structure and functionality between gastroesophageal junction GEJ and the stomach S, with minimal, if any, potential for over-tightening and/or obstructing the gastroesophageal junction GEJ.

### EXAMPLE

### Methods:

Using a porcine explanted stomach, sutures were placed in a pre-determined pattern across the gastric side of the GEJ, using a device and system such as described herein. The endoscope used with the system was held in retroflexion throughout the procedure. Planimetry readings were obtained across the GEJ, before and after plication of the stomach with sutures.

With the explanted porcine stomach, a retroflexion system and device such as described above was used in conjunction with Olympus GIF-H290 and Fujifilm EC760 standard gastroscopes (both from Tokyo, Japan) to place three rows of sutures along the along the greater curve side of the gastro-esophageal junction (such as illustrated in FIG. 1, and as described above). The initial row of sutures was placed in a linear fashion, with the other two rows of sutures placed in a more curvilinear arrangement from both lateral sides of the first row of sutures. The retroflexion device can be rotated (about its longitudinal axis) in retroflexion with the endoscope to/view/access the lesser curve side of the gastroesophageal junction with the two lateral suture rows. It is noted that this part of the stomach is generally less accessible using existing anti-reflux repair systems and devices. The presence of a suturing device provided over the endoscope (as opposed to through the endoscope) is believed to provide a “sizing” function whereby it would not be possible to over-tighten the flap valve while engaging the lesser curve side and tightening the area around the gastroesophageal junction and accentuating the angle of HIS. To demonstrate this, an EndoFLIP 2.0 catheter (325N, manufactured by Medtronic, of Minneapolis, Minnesota, USA) was used to obtain a distensibility index (DI) and cross-sectional area (CSA) readings across the gastroesophageal junction before and after suture plication. The above-described FIG. 3A demonstrates the suture pattern in schematic form, and the above-described FIG. 4, FIG. 4A, FIG. 5, and FIG. 5A demonstrate the suture pattern and effect on the stomach S and gastroesophageal junction GEJ in actual form.

### Results:

Sutures could be placed with minimal technical challenges, with a visual result reminiscent of other ARET's performed by other methods. Planimetry readings did not change after suturing, albeit in an explanted organ, indicating minimal potential for over-tightening at the GEJ.

A promising visual result was obtained at the GEJ (on which FIG. 5 and FIG. 5A are based), with no change in planimetry readings before (DI 19.3 mm²/mmHg and CSA 269 mm²) and after suture placement (DI 18.6 mm²/mmHg and CSA 250 mm²), such as illustrated in FIG. 6. Augmentation of the angle of HIS was demonstrated as expected (such as illustrated in FIG. 5 and FIG. 5A).

### Conclusions:

The results described above could support first-in-human studies. Suture patterns could be developed further, but the approach demonstrated by the above achieves a promising visual result. ARES could be implemented as a novel ARET, given predicate procedures already in clinical practice.

It is expected that the ARES procedure could be implemented as a novel ARET and, given the predicates of similar approaches already in clinical practice or published, first-in-human studies are justified.

Various principles of the present disclosure such as those described above may be implemented in a variety of manners and/or applied to a variety of medical devices, including, but not limited those described above. It will be appreciated that various methods described with respect to the described examples of embodiments may be applied to other embodiments, such as described more broadly/generically above. It is to be understood by one of ordinary skill in the art that the present discussion is a description of illustrative examples of embodiments only, and is not intended as limiting the broader aspects of the present disclosure. Other examples of manners of implementing the disclosed principles will occur to a person of ordinary skill in the art upon reading this disclosure. It should be apparent to those of ordinary skill in the art that variations can be applied to the disclosed methods, and/or to the sequence of steps of the method described herein without departing from the concept, spirit, and scope of the disclosure. It will be appreciated that various features described with respect to one embodiment typically may be applied to another embodiment, whether or not explicitly indicated. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects of methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications used in the practice of the disclosure which may be particularly adapted to specific environments and operative requirements without departing from the principles or spirit or scope of the present disclosure. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element or method, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A method of treating gastroesophageal reflux disease in a patient, said method comprising:
inserting a medical device through the esophagus of the patient and into the stomach of the patient;
retroflexing a distal end of the medical device to access a region of the stomach adjacent the gastroesophageal junction; and
modifying the structure of the region of stomach adjacent the gastroesophageal junction to create a structure which reduces the patient's gastroesophageal reflux disease.

2. The method of claim 1, wherein the medical device is a suturing device, said method further comprising suturing a region of the stomach adjacent the gastroesophageal junction.

3. The method of claim 2, further comprising applying a line of sutures in an anterior-posterior direction and at least one line of sutures in a lateral-medial direction.

4. The method of claim 3, wherein applying at least one line of sutures in a lateral-medial direction further comprises applying a first line of sutures in a lateral-medial direction along a posterior region of the patient's stomach, and a second line of sutures in a lateral-medial direction along an anterior region of the patient's stomach.

5. The method of claim 4, wherein applying at least one line of sutures in a lateral-medial direction further comprises applying at least one line of sutures from a lateral region of the patient's stomach in a direction towards the medial region of the patient's stomach.

6. The method of claim 4, wherein the first line of sutures in a lateral-medial direction is applied before the second line of sutures in a lateral-medial direction is applied.

7. The method of claim 5, wherein applying the line of sutures in an anterior-posterior direction further comprises applying the line of sutures in an anterior-posterior direction along a lateral region of the patient's stomach.

8. The method of claim 3, wherein applying the line of sutures in an anterior-posterior direction further comprises applying the line of sutures in an anterior-posterior direction along a lateral region of the patient's stomach.

9. A method of suturing a patient's stomach to restore the angle of HIS between the patient's stomach and esophagus, said method comprising
inserting a medical device through the esophagus of the patient and into the stomach of the patient;
retroflexing a distal end of the medical device to access a region of the stomach adjacent the gastroesophageal junction; and
modifying the structure of the region of stomach adjacent the gastroesophageal junction to create a structure which reduces the patient's gastroesophageal reflux disease.

10. The method of claim 9, wherein the medical device is a suturing device, said method further comprising suturing a region of the stomach adjacent the gastroesophageal junction.

11. The method of claim 10, further comprising applying a line of sutures in an anterior-posterior direction and at least one line of sutures in a lateral-medial direction.

12. The method of claim 11, wherein applying at least one line of sutures in a lateral-medial direction further comprises applying a first line of sutures in a lateral-medial direction along a posterior region of the patient's stomach, and a second line of sutures in a lateral-medial direction along an anterior region of the patient's stomach.

13. The method of claim 12, wherein applying at least one line of sutures in a lateral-medial direction further comprises applying at least one line of sutures from a lateral region of the patient's stomach in a direction towards the medial region of the patient's stomach.

14. The method of claim 12, wherein the first line of sutures in a lateral-medial direction is applied before the second line of sutures in a lateral-medial direction is applied.

15. The method of claim 13, wherein applying the line of sutures in an anterior-posterior direction further comprises applying the line of sutures in an anterior-posterior direction along a lateral region of the patient's stomach.

16. The method of claim 11, wherein applying the line of sutures in an anterior-posterior direction further comprises applying the line of sutures in an anterior-posterior direction along a lateral region of the patient's stomach.

17. A method for accessing the gastroesophageal junction of a patient's stomach, said method comprising:
inserting a medical device through the esophagus of the patient and into the stomach of the patient; and
retroflexing a distal end of the medical device to access the gastroesophageal junction.

18. The method of claim 17, further comprising axially rotating the medical device to access different sides of the gastroesophageal junction.

19. The method of claim 17, wherein the medical device comprises a medical scope, said method further comprises visually accessing the gastroesophageal junction of the patient by viewing the gastroesophageal junction with the optical system of the medical scope.

20. The method of claim 19, further comprising advancing a medical device in addition to the medical scope along the medical scope to access the gastroesophageal junction of the patient.
